(19) United States
(12) Patent Application Publication
(10) Pub. No.: US 2016/0250435 A1
(43) Pub. Date: Sep. 1, 2016

(54) PACIFIER

(71) Applicants: Andrew Fetterman, Colorado Springs, CO (US); Amanda June Vander Slute, Treasure Island, FL (US)

(72) Inventors: Andrew Fetterman, Colorado Springs, CO (US); Amanda June Vander Slute, Treasure Island, FL (US)

(21) Appl. No.: 14/634,019

(22) Filed: Feb. 27, 2015

(51) Int. Cl. A61M 16/06; A61J 17/00

(52) U.S. Cl. CPC A61M 16/0672; A61J 17/00; A61J 2017/001

(57) ABSTRACT

A pacifier includes a nipple portion, a body portion, and one or more clamps coupled to the body portion configured to attach to a nasal cannula through a friction fit. Each of the clamps is coupled to the body portion of variable lengths to accommodate variable distances between the position of the nasal cannula when inserted at nares and the mouth among different infants and small children. The body portion may further include a handle for handling the pacifier, openings for viewing and to allow substances to pass through. The clamps may further include a hook addition, an extension, and a protrusion addition and may be coupled to the body portion by a trough to facilitate bending of the clamps. The body portion may further include a slanted section to accommodate the nasal cannula.

### BACKGROUND OF THE INVENTION

1. Field of the Invention

The present invention generally relates to a pacifier, and more particularly to a system and device for securing a pacifier.

2. Description of the Related Art

A pacifier, or a soother, is commonly given to an infant or young child to suck upon in the mouth. A pacifier includes a nipple-shaped portion made of rubber, plastic, or silicone that allows the infant to suck upon. A pacifier also includes a mouth shield attached to the nipple-shaped portion that is larger than the mouth of the infant to prevent inadvertent choking or swallowing of the pacifier. Advantages of having an infant sucking on a pacifier include having a soothing effect on the infant, which reduces crying and helps the infant to fall asleep.

There are deficiencies with the related art. For example, a pacifier may be inadvertently released from the infant's mouth (e.g., involuntarily fall out of the infant's mouth when suction or compression pressure on the pacifier is reduced (e.g., the infant is not actively sucking on the pacifier) or actively ejected by the infant (e.g., the infant spits the pacifier out of the mouth)), thereby rendering the pacifier non-functional for its purpose (e.g., soothing the infant). In such a situation, an infant may start producing audible notification (e.g., crying), which a caretaker may be notified that attention is needed and rectify the situation (e.g., replacing the pacifier in the mouth).

However, one problem here is that not all infants have the ability to create an audible sound. In such instances when the caretaker could not be notified within a period of time after the pacifier is released from the infant's mouth to rectify the situation, distress to the infant may ensue. Further, certain infants may be nursing from various medical complications (e.g., a premature infant having various breathing, coronary, or other difficulties). These infants may require assistance from other equipment (e.g., breathing tube/nasal cannula for providing oxygen) until their conditions stabilize. As such, prolonged distress to these infants may be very detrimental to their health when the infant already has pre-existing medical conditions. Irreversible damage (e.g., choking) may result before a caretaker has a chance to be notified (e.g., by machines reading alarming levels of the infant's vital signs) to rectify the situation.

### SUMMARY OF THE INVENTION

Accordingly, the invention is directed to a pacifier and method of using and securing the pacifier that substantially obviates one or more of the problems due to limitations and disadvantages of the related art.

An advantage of an embodiment is to maintain and secure the pacifier in an infant's mouth even when the infant has released suction and compression pressure on the pacifier.

Another advantage of an embodiment is to aid in maintaining the infant's demeanor and keep the infant from elevating his/her emotional state, blood pressure, or other biological states, which may cause additional strain on respiratory, circulatory, or other medically significant processes. Avoiding these unwanted effects contributes to the infant's well-being and improves the level of care the infant would receive.

Another advantage of an embodiment is to provide an opportunity for relief to an infant wearer of the pacifier from other discomforts such as Tender Grips, tape or other securing substances that would cause strain to the surface skin of the cheeks by obviating the need to use such substances for securing the pacifier or the nasal cannula.

Another advantage of an embodiment is to promote natural suction and feeding instincts of the infant wearer of the pacifier in order to promote in the optimal development of the infant by maintaining similarities to a nipple of a human female, which the infant instinctively would suck on as an instinctive response.

Yet another advantage of an embodiment is to ease the transition from an intubation tube to independent breathing, which may additionally reduce the time required for intubation. It is recognized that some infants may have a need to and have used an intubation tube (a tube which is inserted into the infant's throat for providing oxygen to the lungs and assisting the breathing process (e.g., for infants with certain conditions)), which undoubtedly have experienced significant discomfort resulting in elevated vital signs (e.g., heightened heart rate). The pacifier provides an opportunity to provide relief and calm the infant and to provide a positive stimulation for reversing and controlling the infant's elevated vital signs.

A further advantage of an embodiment is to promote breathing through the nares (nostrils) as opposed to the mouth, which benefits the infant by increasing oxygen saturation and delivery of oxygen to the blood.

Additional features and advantages of the invention will be set forth in the description which follows, and in art will be apparent from the description, or may be learned by practice of the invention. The objectives and other advantages of the invention will be realized and attained by the structure particularly pointed out in the written description and claims hereof as well as the appended figures.

According to an embodiment, a pacifier includes a nipple portion, a body portion coupled to the nipple portion, and a clamp coupled to a side of the body portion. The pacifier further includes one or more clamps coupled to respective one or more sides of the body portion. One of the sides of the body portion is longer than another of the sides of the body portion. The pacifier further includes a handle coupled to the body portion. The body portion includes one or more openings. The clamp is configured to attach a nasal cannula. The nipple portion is configured to fit into a mouth of an infant or small child when the clamp is attached to the nasal cannula that is fitted into nares of the infant. The clamp includes one or more of a hook addition to an inner arc portion of the clamp, an extension to a top portion of the clamp, and a protrusion addition to and outer arc portion of the clamp. The pacifier further includes a trough at a coupling of the clamp and the body portion. The pacifier further includes a slanted section at a section of the body portion proximate to the clamp and opposite of the nipple portion.

According to an embodiment, a method of fitting a pacifier includes fitting a nipple portion of the pacifier into a mouth of an infant or small child and attaching a nasal cannula into a clamp coupled to a body portion of the pacifier.

According to an embodiment, a pacifier assembly includes a pacifier including a nipple portion, a body portion coupled to the nipple portion, and a clamp coupled to a side of the body portion and a nasal cannula attached to the clamp. The pacifier further includes one or more clamps coupled to respective one or more sides of the body portion. The pacifier further includes a handle coupled to the body portion. One of the sides of the body portion is longer than another of the sides of the body portion. The body portion comprises one or more openings. The nipple portion is configured to fit into a mouth of an infant or small child when the clamp is attached to the nasal cannula fitted into nares of the infant. The clamp includes one or more of a hook addition to an inner arc portion of the clamp, an extension to a top portion of the clamp, and a protrusion addition to and outer arc portion of the clamp. The pacifier further includes a trough at a coupling of the clamp and the body portion. The pacifier further includes a slanted section at a section of the body portion proximate to the clamp and opposite of the nipple portion.

It is to be understood that both the foregoing general description and the following detailed description are exemplary and explanatory and are intended to provide further explanation of the invention as claimed.

The phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

It shall be understood that the term “means,” as used herein, shall be given its broadest possible interpretation in accordance with 35 U.S.C., Section 112(f). Accordingly, a claim incorporating the term “means” shall cover all structures, materials, or acts set forth herein, and all of the equivalents thereof. Further, the structures, materials or acts and the equivalents thereof shall include all those described in the summary of the invention, brief description of the drawings, detailed description, abstract, and claims themselves.

The preceding is a simplified summary of the disclosure to provide an understanding of some aspects of the disclosure. This summary is neither an extensive nor exhaustive overview of the disclosure and its various aspects, embodiments, and/or configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and/or configurations of the disclosure are possible, utilizing, alone or in combination, one or more of the features set forth above or described in detail below.

### BRIEF DESCRIPTION OF THE FIGURES

The accompanying figures, which are included to provide a further understanding of the invention are incorporated in and constitute a part of this specification, illustrate embodiments of the invention and together with the description serve to explain the principles of the invention.

FIG. 1A illustrates a side view of a pacifier according to an embodiment; FIG. 1B illustrates a back view of a pacifier according to an embodiment;

FIG. 2 illustrates a view of an infant wearing a pacifier according to an embodiment; and

FIG. 3A-3C illustrate views of clamp attachments of a pacifier according to an embodiment.

### DETAILED DESCRIPTION OF THE PREFERRED EMBODIMENTS

Reference will now be made in additional detail to an embodiment of the present invention, example of which is illustrated in the accompanying figures.

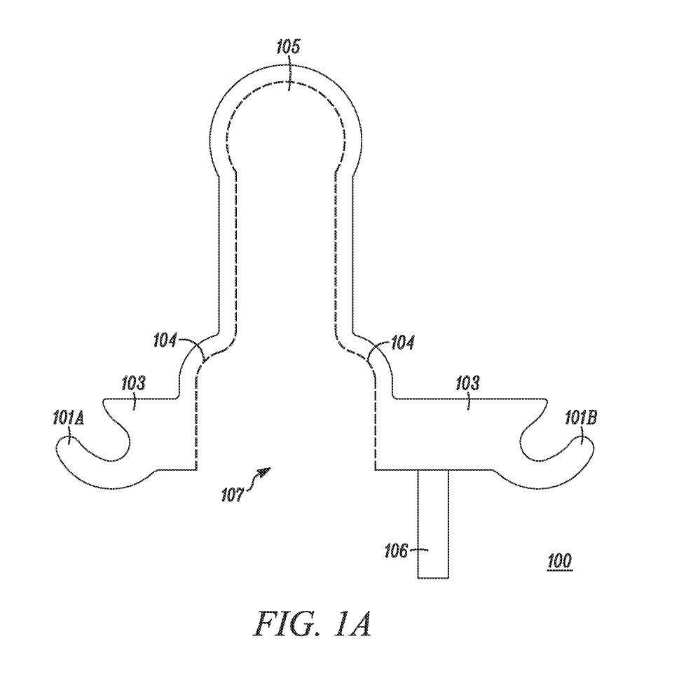

FIG. 1A illustrates a side view of a pacifier according to an embodiment; FIG. 1B illustrates a front view of a pacifier according to an embodiment.

Referring to FIGS. 1A and 1B, a pacifier 100 includes a body portion 103 attached to a nipple portion 105 through a curved portion 104. The pacifier 100 also includes one or more of clamps 101A and 101B and handle 106 attached to the body portion 103.

In an embodiment, the nipple portion 105 extends from the body portion 103 and is configured to be fitted into the mouth (e.g., the mouths of infants and small children). The body portion 103 surrounds the nipple portion 105 through the curved portion 104; the body portion 103 have a left, right, top and bottom and acts as a shield from which the lips and skin outside of the mouth meets, which limits the distance from which the nipple portion 105 can be inserted into the mouth. For example, in an embodiment, the nipple portion 105 (including the curved portion 104) may extend 29 mm (e.g., a typical size of a pacifier for an infant or small children). In an embodiment, the design of the nipple portion 105 is sealed and does not allow air to be drawn through in order to prevent internal distress, bloat, colic, or other medical concerns relating to ingesting air. Further, the nipple portion 105 may have a shape similar to the female nipple and anatomy.

Further referring to the body portion 103, in an embodiment, the clamps 101A and 101B are positioned at the top and bottom of the body portion 103. The clamps 101A and 101B (or clasps) are configured to attach to a tube-shaped object (e.g., a nasal cannula) for securing the pacifier to the object. In an embodiment, the clamps 101A and 101B may have a “U”-shaped insert design, separated finger style clasps, or other variations of insert clamps. The clamps 101A and 101B allow positioning, fixture, and alignment of the pacifier 100 to the tube-shaped object.

In an embodiment, the attachment of the clamps 101A and 101B to the nasal cannula, once inserted, may be maintained by a friction fit (e.g., through friction between the coupled surfaces of the nasal cannula and the clamps 101A and 101B) or other type of fits as now known or may be later derived. In the removal of the clamps 101A and 101B from the nasal cannula, direct force or pressure to the pacifier 101 may be applied in a direction opposite from the nasal cannula. The amount of force or pressure to remove the pacifier 100 from the nasal cannula may depend on the tightness of the fit. In a preferred embodiment, the tightness of the fit is enough such that the pacifier 100 may only be removed by an intentional force applied by an adult (or a patient or an infant) (e.g., a swat, swipe, or pull on the pacifier 100 to release it from the nasal cannula), but not by an unintentional or accidental force applied by an infant or small child. However, the fit should not be as tight to impede or compress the nasal cannula hosing or alter the delivery of oxygen through the nasal cannula but instead should simply maintain the location of the pacifier 100 in respect to the location of the nasal cannula. In an embodiment, the clamps 101A and 101B may be made of plastic, silicone, metal, or other flexible materials that could provide the insertion, removal, and coupling characteristics as needed by an intended application (e.g., fitted on a nasal cannula).

In an embodiment, when the infant is wearing a nasal cannula, the clamps 101A or 101B may be attached to the nasal cannula such that the mouth opening of the infant is in alignment with the pacifier 100 (e.g., the resulting position of pacifier 100 is directly over the mouth and the nipple portion 105 may be inserted into the mouth opening). For example, the length of the body portion 103 between one of the clamps 101A and 101B and the curved portion 104 (where the curved portion 104 and the nipple portion 105 may be inserted into the infant's mouth) may be between 3 mm to 35 mm (e.g., typical range of distances between fitting the clamps 101A and 101B by an infant wearing a nasal cannula and the top of the mouth of the infant). In an embodiment, a pacifier 100 includes both clamps 101A and 101B, where the body portion 103 between the clamp 101A and the curved portion 104 is at one length (e.g., 5 mm) and the body portion 103 between the clamp 101A and the curved portion 104 is at another length (e.g., 25 mm) such that the pacifier 100 may facilitate infants and nasal cannula of different sizes (e.g., either one of clamps 101A or 101B may be chosen to fit for different infants in the same pacifier 100 (e.g., by turning the pacifier 100 such that the chosen clamp is fitted to the nasal cannular for a particular infant)). In a further embodiment, other portions of the body portion 103 (e.g., the left and right) may also include other clamps and the corresponding connecting parts of the body portion 103 may be of other lengths.

In other variations, the clamps 101A and 101B may include varying sizes and shapes of clamps, angles of clamps, and heights of clamps 101A and 101B as measured from the base of the curved portion 104 or the nipple portion 105 where the clamps 101A and 101B attach to the body portion 103. For example, in a preferred embodiment, variations of clasp angles may be anywhere from 15-70 degrees from the flat face of the body portion 103 in the direction of the nipple portion 105.

In an embodiment, the body portion 103 may include an indentation or trough 112 configured to allow further flexing and pivoting of the clasps 101A and 101B (e.g., the clasps 101A and 101B has greater ability to bend at the site of the trough 112). The body portion 103 may also include a slanted section 113 at a back side of the body portion 103 (e.g., the portion opposite of the nipple portion 105) configured for an ideal positioning and alignment of the nasal cannula when attached to the clasps 101A and 101B.

In an embodiment, the body portion 103 may include openings 114 configured to act as airways, portions for expelling fluids, and windows for viewing and monitoring the infant's skin tone when wearing the pacifier. For example, the openings 114 allow for the release and expulsion of fluids from the child's mouth. The openings 114 further allow a window of visibility to monitor the skin and lips. In the event of discolorations of the lips or skin of the infant. Accordingly, identification can be made with a direct line of visibility free from obstructions and material housings of the pacifier 100.

In an embodiment, the body portion 103 may include a handle 106 configured to allow one (e.g., an adult) to administer the pacifier 100 without needing to touch or contaminate portions of the pacifier 100 that would be inserted into the infant's mouth. The body portion 103 may also include a hollow or filled back-side 107 of the nipple portion 105.

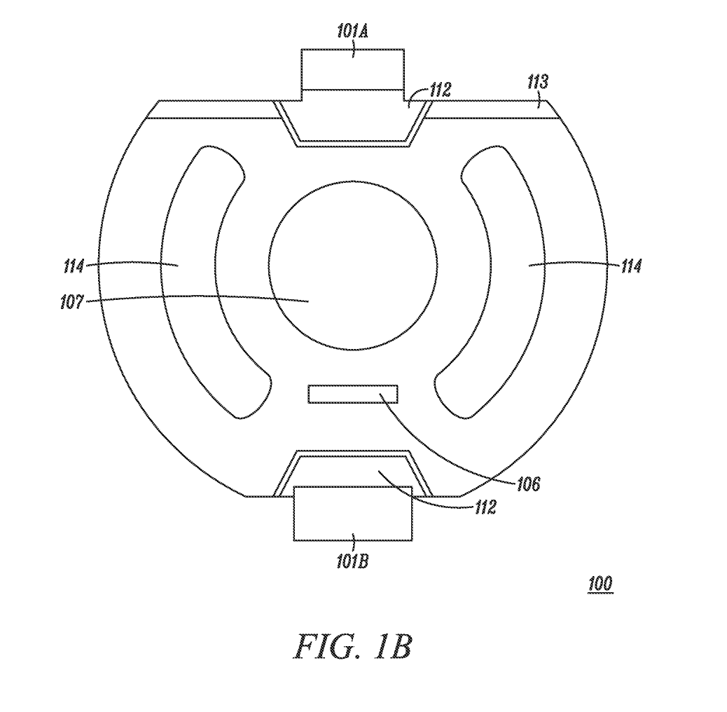

FIG. 2 illustrates a view of an infant wearing a pacifier according to an embodiment.

Referring to FIG. 2, the pacifier 100, when coupled to the nasal cannula 122, allows an infant 121 to release suction and compression pressure (e.g., when the infant is not actively sucking on the pacifier) while maintaining and securing its position in the infant's mouth when the pacifier 100 is inserted into the infant's mouth and held in place through the coupling to the nasal cannula 122.

An application of the pacifier 100 is to maintain the infant's demeanor and keep the infant from elevating his/her emotional state, blood pressure, or other biological states, which may cause additional strain on respiratory, circulatory, or other medically significant processes. Avoiding these unwanted effects contributes to the infant's well-being and improves the level of care the infant would receive.

Another application of an embodiment is that it provides an opportunity for relief to an infant wearer of the pacifier 100 from other discomforts. For example, in an alternative method, Tender Grips may be used for securing the nasal cannula 121 (e.g., sticky pads are applied to the upper cheeks for holding the nasal cannula 121 in place). However, such a method applies a strain to the surface skin of the cheeks, which causes the surface skin to potentially break down, thereby developing a rash and tear to the skin. Another alternative of using tape tends to have the same result as with the Tender Grips as described above.

In an embodiment, the pacifier 100 is configured to hold the nasal cannula 121 in a fixed position and obviates the need of using Tender Grips, tape, or other securing substances. By relieving the skin of the Tender Grips, tape, or other securing substances, the infant is shielded from the tearing effects to the skins from these substances. For infants who have developed a rash or tear in the skin in the application areas, the infant may now heal these areas. As such, a higher level of care may be provided, which also induces increased infant/guardian satisfaction. Further, overall healing time to the affected areas may be reduced.

In another exemplary application of the pacifier 100, constant contact and correct placement of nasal cannula 121 may be realized in the infant's nares (nostrils) for maximum benefit (e.g., oxygen delivery). For example, when the pacifier 100 is inserted into the infant's mouth, the attached (through the clamps 101A and 101B of the pacifier 100) nasal cannula 121 (which is correspondingly inserted into the nares) is held in place by the stabilized pacifier 100 in the mouth (similar to the pacifier 100 being stabilized by the nasal cannula 121 as discussed above).

In yet another application, the pacifier 100 also promotes natural suction and feeding instincts of the infant wearer to promote in the optimal development of the infant. For example, the pacifier 100 advances the well-being of the infant by maintaining similarities of a nipple of a human female, which the infant instinctively would suck on. In an embodiment, the dimensions of the nipple portion 105 would be based off of the nipple of a human female in order to induce this instinctive response from the infant.

In another application, it is recognized that some infants may have a need to and have used an intubation tube (a tube which is inserted into the infant's throat for providing oxygen to the lungs and assisting the breathing process (e.g., for infants with certain conditions)). Once this intubation tube is removed, the nasal cannula 121 is applied to continue the delivery of oxygen to the lungs. At this point, the infant will undoubtedly have experienced significant discomfort resulting in elevated vital signs (e.g., heightened heart rate). In an embodiment, the pacifier 100 provides an opportunity to provide relief and calm the infant and to provide a positive stimulation for reversing and controlling the infant's elevated vital signs. As such, the infant may be eased into the transition from intubation to independent breathing, which may additionally reduce the time required for intubation.

In a further application, the use of the pacifier promotes breathing through the nares (nostrils) as opposed to the mouth, which benefits the infant by increasing oxygen saturation and delivery of oxygen to the blood.

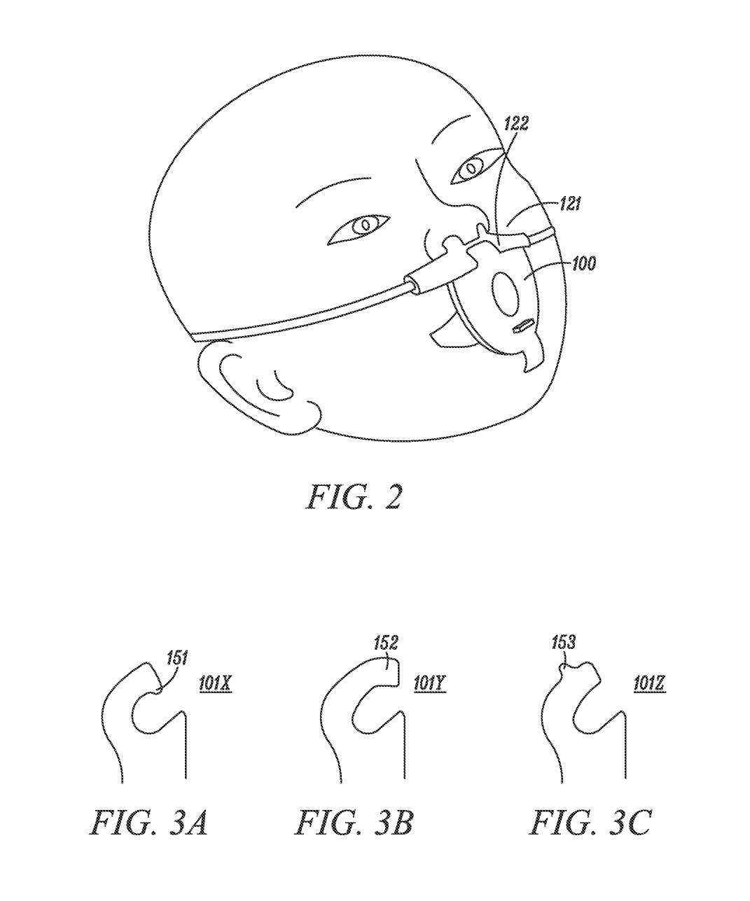

FIG. 3A-3C illustrate views of clamp attachments of a pacifier according to an embodiment.

Referring to FIG. 3A, clamp 101X includes a hook addition 151 to an inner arc portion of the clamp 101X. When the clamp 101X is attached to a nasal cannula, the hook addition 151 provides additional pressure for holding the nasal cannula in position within the clamp 101X and avoids unintended removal of the nasal cannula.

Referring to FIG. 3B, clamp 101Y includes an extension 152 to a top portion of the clamp 101Y. In an embodiment, the extension 152 may include a rounding arc extending the top portion of the clamp 101Y closer to the body portion 103 of the pacifier 100. In an application, the extension 152 is configured to provide additional pressure for holding the nasal cannula in position within the clamp 101Y and avoids unintended removal of the nasal cannula.

Referring to FIG. 3C, clamp 101Z includes a protrusion addition 153 to an outer arc portion of the clamp 101Z. In an application, an individual may apply pressure to a front portion of the protrusion addition 152 (e.g., pressing with a thumb) to enlarge the opening of the clamp 101Z for an easier insertion of a nasal cannula to the opening of the clamp 101Z.

In an embodiment, clamps 101A and 101B may include elements of clamps 101X, 101Y, and 101Z in one or more combinations or with other features of clamps as known now or may be later derived.

The present disclosure, in various aspects, embodiments, and/or configurations, includes components, methods, processes, systems and/or apparatus substantially as depicted and described herein, including various aspects, embodiments, configurations embodiments, subcombinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and/or reducing cost of implementation.

The foregoing discussion has been presented for purposes of illustration and description. The foregoing is not intended to limit the disclosure to the form or forms disclosed herein. In the foregoing description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this description, with each claim standing on its own as a separate preferred embodiment of the disclosure.

Moreover, though the description has included a description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A pacifier, comprising:
a nipple portion;
a body portion coupled to the nipple portion; and
a clamp coupled to a side of the body portion.

2. The pacifier of claim 1, further comprising one or more clamps coupled to respective one or more sides of the body portion.

3. The pacifier of claim 1, further comprising a handle coupled to the body portion.

4. The pacifier of claim 2, wherein one of the sides of the body portion is longer than another of the sides of the body portion.

5. The pacifier of claim 1, wherein the body portion comprises one or more openings.

6. The pacifier of claim 1, wherein the clamp is configured to attach a nasal cannula.

7. The pacifier of claim 6, wherein the nipple portion is configured to fit into a mouth of an infant or small child when the clamp is attached to the nasal cannula that is fitted into nares of the infant.

8. The pacifier of claim 1, wherein the clamp comprises one or more of a hook addition to an inner arc portion of the clamp, an extension to a top portion of the clamp, and a protrusion addition to an outer arc portion of the clamp.

9. The pacifier of claim 1, further comprising a trough at a coupling of the clamp and the body portion.

10. The pacifier of claim 1, further comprising a slanted section at a section of the body portion proximate to the clamp and opposite of the nipple portion.

11. A method of fitting a pacifier, comprising:
fitting a nipple portion of the pacifier into a mouth of an infant or small child; and
attaching a nasal cannula into a clamp coupled to a body portion of the pacifier.

12. A pacifier assembly, comprising:
a pacifier comprising a nipple portion; a body portion coupled to the nipple portion; and
a clamp coupled to a side of the body portion; and
a nasal cannula attached to the clamp.

13. The pacifier assembly of claim 12, wherein the pacifier further comprises one or more clamps coupled to respective one or more sides of the body portion.

14. The pacifier assembly of claim 12, wherein the pacifier further comprises a handle coupled to the body portion.

15. The pacifier assembly of claim 13, wherein one of the sides of the body portion is longer than another of the sides of the body portion.

16. The pacifier assembly of claim 12, wherein the body portion comprises one or more openings.

17. The pacifier assembly of claim 12, wherein the nipple portion is configured to fit into a mouth of an infant or small child when the clamp is attached to the nasal cannula fitted into nares of the infant.

18. The pacifier assembly of claim 12, wherein the clamp comprises one or more of a hook addition to an inner arc portion of the clamp, an extension to a top portion of the clamp, and a protrusion addition to an outer arc portion of the clamp.

19. The pacifier assembly of claim 12, wherein the pacifier further comprises a trough at a coupling of the clamp and the body portion.

20. The pacifier assembly of claim 12, wherein the pacifier further comprises a slanted section at a section of the body portion proximate to the clamp and opposite of the nipple portion.
